SEQUENCE LISTING (26 sequences)

SEQ ID NO: 1 - DNA, 963 bp, Homo sapiens
atgtctaagtcagagtctcctaaagagcccgaacagctgaggaagctcttcattggagggttgagctttgaaacaactgatgagagcctgaggagccattttgagcaatggggaacgctcacggactgtgtggtaatgagagatccaaacaccaagcgctccaggggctttgggtttgtcacatatgccactgtggaggaggtggatgcagctatgaatgcaaggccacacaaggtggatggaagagttgtggaaccaaagagagctgtctccagagaagattctcaaagaccaggtgcccacttaactgtgaaaaagatatttgttggtggcattaaagaagacactgaagaacatcacctaagagattattttgaacagtatggaaaaattgaagtgattgaaatcatgactgaccgaggcagtggcaagaaaaggggctttgcctttgtaacctttgacgaccatgactccgtggataagattgtcattcagaaataccatactgtgaatggccacaactgtgaagttagaaaagccctgtcaaagcaagagatggctagtgcttcatccagccaaagaggtcgaagtggttctggaaactttggtggtggtcgtggaggtggtttcggtgggaatgacaacttcggtcgtggaggaaacttcagtggtcgtggtggctttggtggcagccgtggtggtggtggatatggtggcagtggggatggctataatggatttggtaatgatggaagcaattttggaggtggtggaagctacaatgattttgggaattacaacaatcagtcttcaaattttggacccatgaagggaggaaattttggaggcagaagctctggcccctatggcggtggaggccaatactttgcaaaaccacgaaaccaaggtggctatggcggttccagcagcagcagtagctatggcagtggcagaagattttaa

SEQ ID NO: 2 - DNA, 1245 bp, Homo sapiens
atgtctgaatatattcgggtaaccgaagatgagaacgatgagcccattgaaataccatcggaagacgatgggacggtgctgctctccacggttacagcccagtttccaggggcgtgtgggcttcgctacaggaatccagtgtctcagtgtatgagaggtgtccggctggtagaaggaattctgcatgccccagatgctggctggggaaatctggtgtatgttgtcaactatccaaaagataacaaaagaaaaatggatgagacagatgcttcatcagcagtgaaagtgaaaagagcagtccagaaaacatccgatttaatagtgttgggtctcccatggaaaacaaccgaacaggacctgaaagagtattttagtacctttggagaagttcttatggtgcaggtcaagaaagatcttaagactggtcattcaaaggggtttggctttgttcgttttacggaatatgaaacacaagtgaaagtaatgtcacagcgacatatgatagatggacgatggtgtgactgcaaacttcctaattctaagcaaagccaagatgagcctttgagaagcagaaaagtgtttgtggggcgctgtacagaggacatgactgaggatgagctgcgggagttcttctctcagtacggggatgtgatggatgtcttcatccccaagccattcagggcctttgcctttgttacatttgcagatgatcagattgcgcagtctctttgtggagaggacttgatcattaaaggaatcagcgttcatatatccaatgccgaacctaagcacaatagcaatagacagttagaaagaagtggaagatttggtggtaatccaggtggctttgggaatcagggtggatttggtaatagcagagggggtggagctggtttgggaaacaatcaaggtagtaatatgggtggtgggatgaactttggtgcgttcagcattaatccagccatgatggctgccgcccaggcagcactacagagcagttggggtatgatgggcatgttagccagccagcagaaccagtcaggcccatcgggtaataaccaaaaccaaggcaacatgcagagggagccaaaccaggccttcggttctggaaataactcttatagtggctctaattctggtgcagcaattggttggggatcagcatccaatgcagggtcgggcagtggttttaatggaggctttggctcaagcatggattctaagtcttctggctggggaatgtag

SEQ ID NO: 3 - DNA, 52 bp, Artificial Sequence
  The forward primer of hnRNP A1
catcattttggcaaagaattccaccatgtctaagtcagagtctcctaaagag

SEQ ID NO: 4 - DNA, 69 bp, Artificial Sequence
  The reverse primer of hnRNP A1
gctccccgggggtacctcgagctacttgtcatcgtcgtccttgtagaaatcttctgccactgccatagc

SEQ ID NO: 5 - DNA, 21 bp, Artificial Sequence
  small interfering RNA-Ctrl
uucuccgaacgugucacgutt

SEQ ID NO: 6 - DNA, 21 bp, Artificial Sequence
  small interfering RNA-hPARG
gcggugaaguuagauuacatt

SEQ ID NO: 7 - DNA, 48 bp, Artificial Sequence
  The forward primer of TDP-43
catcattttggcaaagaattccaccatgtctgaatatattcgggtaac

SEQ ID NO: 8 - DNA, 72 bp, Artificial Sequence
  The reverse primer of TDP-43
gctccccgggggtacctcgagttaagcgtagtctgggacgtcgtatgggtacattccccagccagaagactt

SEQ ID NO: 9 - DNA, 36 bp, Artificial Sequence
  The forward primer of hnRNP A1-K298A
ctttgcagcaccacgaaaccaaggtggctatggcgg

SEQ ID NO: 10 - DNA, 39 bp, Artificial Sequence
  The reverse primer of hnRNP A1-K298A
ttcgtggtgctgcaaagtattggcctccaccgccatagg

SEQ ID NO: 11 - DNA, 62 bp, Artificial Sequence
  The forward primer of hnRNP A1-PBMmut
aggtgcccacttaactgtggttgttatatttgttggtggcattaaagaagacactgaagaac

SEQ ID NO: 12 - DNA, 51 bp, Artificial Sequence
  The reverse primer of hnRNP A1-PBMmut
ccaccaacaaatataacaaccacagttaagtgggcacctggtctttgagaa

SEQ ID NO: 13 - DNA, 36 bp, Artificial Sequence
  The forward primer of TDP-43(1-274)
atggccatggaggccgaattcatgtctgaatatatt

SEQ ID NO: 14 - DNA, 47 bp, Artificial Sequence
  The reverse primer of TDP-43(1-274)
catgtctggatccccgcggccgcctaacttctttctaactgtctatt

SEQ ID NO: 15 - DNA, 42 bp, Artificial Sequence
  The forward primer of TDP-43(274-414)
cagcaaatgggtcgcgccaccggatccggaagatttggtggt

SEQ ID NO: 16 - DNA, 36 bp, Artificial Sequence
  The reverse primer of TDP-43(274-414)
gtggtggtggtggtgctcgagcattccccagccaga

SEQ ID NO: 17 - DNA, 21 bp, Artificial Sequence
  small interfering RNA-mPARG
gcaguuucuuacaccuauatt

SEQ ID NO: 18 - DNA, 21 bp, Artificial Sequence
  small interfering RNA-mPARP1
cgacgcuuauuacuguacutt

SEQ ID NO: 19 - DNA, 20 bp, Artificial Sequence
  The forward primer of mParg
agcctctgacacgcttacac

SEQ ID NO: 20 - DNA, 20 bp, Artificial Sequence
  The reverse primer of mParg
cagtcacaccacctccaaca

SEQ ID NO: 21 - DNA, 20 bp, Artificial Sequence
  The forward primer of mGAPDH
caccatcttccaggagcgag

SEQ ID NO: 22 - DNA, 22 bp, Artificial Sequence
  The reverse primer of mGAPDH
ccttctccatggtggtgaagac

SEQ ID NO: 23 - DNA, 20 bp, Artificial Sequence
  The forward primer of dParp
atgaagtacggaggccaacc

SEQ ID NO: 24 - DNA, 20 bp, Artificial Sequence
  The reverse primer of dParp
tcttcacctgacgcaaacca

SEQ ID NO: 25 - DNA, 20 bp, Artificial Sequence
  The forward primer of dActin
gagcgcggttactctttcac

SEQ ID NO: 26 - DNA, 20 bp, Artificial Sequence
  The reverse primer of dActin
gccatctcctgctcaaagtc

(19) United States
(12) Patent Application Publication
(10) Pub. No.: US 2022/0110936 A1
(43) Pub. Date: Apr. 14, 2022

(54) PHARMACEUTICAL COMPOSITION FOR TREATMENT OF NEURODEGENERATIVE DISEASES OR DISEASES CAUSED BY ABNORMALITY OF RNA BINDING PROTEIN AND APPLICATIONS THEREOF

(71) Applicant: Shanghai Institute of Organic Chemistry, Chinese Academy of Sciences, Shanghai (CN)

(72) Inventors: Yanshan FANG, Shanghai (CN); Yongjia DUAN, Shanghai (CN)

(21) Appl. No.: 17/427,404

(22) PCT Filed: Feb. 2, 2019
(86) PCT No.: PCT/CN2019/074579

§ 371 Date: Jul. 30, 2021

(51) Int. Cl. A61K 31/502; A61P 25/28

(52) U.S. Cl. CPC A61K 31/502; A61P 25/28

(57) ABSTRACT

Disclosed are a pharmaceutical composition for treatment of neurodegenerative diseases or diseases caused by abnormality of RNA binding protein and applications thereof, in particular the application in the treatment of ALS. The pharmaceutical composition can significantly enhance the dynamic performance of stress particles containing RNA binding proteins such as hnRNP A1 and TDP-43 proteins; influences the interaction between the RNA binding proteins and other poly ADP ribosylation modified proteins or other PAR binding proteins; influences the subcellular localization and stress response of RNA binding proteins; influences the liquid-liquid phase separation and aggregation tendency of RNA binding proteins; influences the co-phase separation between RNA binding proteins; influences the interaction of RNA binding proteins in cells; and has a significant inhibitory effect on neurotoxicity caused by RNA binding proteins.

### TECHNICAL FIELD

The present invention relates to a pharmaceutical composition for treatment of a neurodegenerative disease or diseases caused by abnormality of RNA-binding proteins and applications thereof.

### BACKGROUND ARTS

1. Amyotrophic Lateral Sclerosis (ALS)

ALS overview: ALS is a neurological disease caused by the degeneration of motor neurons, with an average incidence of 1-2 per 100,000 people (Bento-Abreu et al., 2010). ALS is mainly manifested by a massive loss of descending motor neurons (including brain stem and ventral horn) and ascending motor neurons (including cortex, etc.). The main symptoms include: muscle atrophy, muscle spasm, dysarthria, dysphagia, loss of basic motor abilities in later stages and eventually death due to respiratory failure (Mitchell and Borasio, 2007; Nguyen et al., 2018; van Es et al., 2017).

Current status of ALS treatment and drug development: ALS is still incurable. Currently, only Riluzole and Edaravone are approved as clinical drugs by the US Food and Drug Administration (FDA). Riluzole slows the progression of ALS by inhibiting glutamatergic activity, but it has poor specificity and only prolongs the patient's life by 3-6 months, with side effects such as nausea, dizziness and even pneumonia (Lacomblez et al., 1996; Tripathi and Al-Chalabi, 2008). Edaravone, a free radical scavenger, was approved by the FDA in 2017 for treatment of ALS. The result of phase III clinical trials showed that Edaravone was able to alleviate some degree of physical impairment in ALS patients, but the drug was only effective in a small percentage of patients who met the criteria for post-hoc analysis (Abe et al., 2017).

The known pathogenesis of ALS: The pathogenesis of ALS is complex, and the exact molecular mechanism remains unclear, but the most common ALS is more due to mutations in RNA-binding proteins. In normal cells, RNA-binding proteins function by binding to RNA to form ribonucleoprotein (RNP) complexes. In the presence of cellular stress, RNP complexes form stress granules to maintain cellular homeostasis (Buchan et al., 2009). Mutations in the genes encoding these proteins can affect the formation and function of RNP granules and contribute to their abnormal aggregation, which is an important pathological hallmark of many neurodegenerative diseases including ALS. There are hundreds of intracellular RNA-binding proteins, and the main RNA-binding proteins known to be associated with ALS include heterogeneous nuclear ribonucleoprotein A1 (hnRNP A1), Transactivation response element DNA-binding protein 43 (TDP-43), Fused in sarcoma (FUS), TATA-binding protein-associated factor 15 (TAF15), Ewing sarcoma breakpoint region 1 (EWSR1), T-cell intracytoplasmic antigen 1 (TIA-1), etc. (Harrison and Shorter, 2017).

ALS major pathogenic proteins hnRNP A1 and TDP-43: hnRNP A1 and TDP-43 are mainly localized in the nucleus under normal physiological state and are involved in the regulation of various cellular physiological functions. Regarding the molecular mechanisms by which they contribute to ALS, what is known includes (1) aberrant post-translational modifications of hnRNP A1 and TDP-43 proteins, such as hyperphosphorylation, ubiquitination, etc. and abnormal intracellular aggregation (Neumann et al., 2006). (2) In ALS patient tissues, both hnRNP A1 and TDP-43 proteins were found to be aberrantly localized in the cell cytoplasm and formed pathological inclusion bodies (Kim et al., 2013; Neumann et al., 2006). (3) Aberrant formation and regulation of stress granules in which hnRNP A1 and TDP-43 are involved: both hnRNP A1 and TDP-43 proteins are important components of cellular stress granules; mutations in hnRNP A1 and TDP-43 can affect their ability to localize to and bind to stress granules; accordingly, regulation of stress granule formation can alleviate neurotoxicity due to these RNA-binding proteins (Elden et al., 2010; Liu-Yesucevitz et al., 2010; Li et al., 2013; Kim et al., 2013; Kim et al., 2014; Naruse et al., 2018). In addition, the molecular mechanisms by which hnRNP Al and TDP-43 contribute to the development of ALS are also related to their abnormal functions in regulating RNA processing and their abnormal effects on mitochondria.

Relationship between LLPS and ALS: RNA-binding proteins such as hnRNP A1 are able to interact with RNA to form RNP granules, especially to form cellular stress granules. Recent studies have shown that these RNA-binding proteins are able to form droplets similar to cellular stress granules by liquid-liquid phase separation (LLPS) in in vitro experiments (Lin et al., 2015). These droplets are highly dynamics and reversible, and can fuse or separate from other droplets around them. When the external environmental conditions change, the droplets formed via LLPS continuously solidify and gradually form amyloid aggregates (Hyman et al., 2014; Lin et al., 2015; Molliex et al., 2015; Wang et al., 2014). This process is similar to the progressive loss of fluidity and reversibility, and eventually development of irreversible pathological protein inclusion bodies of stress granules in vivo during the progress of ALS, and thus can be used to characterize the aggregation tendency of RNA-binding proteins.

2. Poly(ADP-Ribosyl)ation, PARylation

PARylation overview: The reversible reaction of protein PARylation modification is catalyzed by poly(ADP-ribose) polymerase (PARP) family and poly(ADP-ribose) glycohydrolase (PARG). The main cellular functions that PARylation modifications participate in regulating are: (1) regulating chromatin structure and gene transcription; (2) assisting the assembly of DNA damage repair complexes and promoting their functions; (3) activating the proteasome to remove damaged histones; (4) promoting protein translocation into the Cajal body; (5) Affecting intra- and extra-nuclear protein translocations; (6) promoting release of apoptosis inducing factor (AIF) from mitochondria and its transport to the nucleus; (7) regulating the assembly and normal function of stress granules (Luo and Kraus, 2012).

Clinical applications and drug development of PARP inhibitors: The clinical applications of PARP inhibitors are mainly focused on oncology treatment, such as PARP1 inhibitor Olaparib was approved by FDA for treatment of ovarian, breast and prostate cancers associated with BRCA1 and BRCA2 gene mutations (Fong et al., 2009; Tutt et al., 2010). In addition, a few laboratory and preclinical studies suggest a neuroprotective effect of reducing PARylation levels for conditions such as Huntingtonian chorea, local ischemia and axonal injury in the brain (Brochier et al., 2015; Cardinale et al., 2015; Egi et al., 2011; Teng et al., 2016). However, there are no relevant studies or reports on the use of PARP inhibitors such as Olaparib in treatment of diseases such as ALS.

3. PARylation and ALS

Little has been reported about the role of PARylation in ALS until now, with only one recent paper mentioning that free intracellular poly(ADP-ribose) (PAR) may bind to TDP-43 and affect its localization to stress granules (McGurk et al., 2018). However, McGurk et al. only investigated the effect of non-covalent PAR-binding on TDP-43, a solitary pathogenic protein, and only observed that the binding of TDP-43 to PAR chains affects its localization to cellular stress granules; McGurk et al. suggested that Tankyrase (i.e., PARP5) is the main enzyme catalyzing PARylation modification of TDP-43, and Tankyrase is mainly localized in the cell cytoplasm. The Tankyrase inhibitors XAV939 and G007-LK used in the study of McGurk et al. were not FDA-approved clinical agents, but only laboratory inhibitors for research; while neither efficacy nor safety experiment of XAV939 and G007-LK were conducted to inhibit TDP-43-induced neurotoxicity in their study.

### Content of the Present Invention

The technical problem to be solved by the present invention is to overcome the lack of effective drugs for treating neurodegenerative diseases or diseases caused by abnormality of RNA-binding proteins in the prior art, and the present invention provide a pharmaceutical composition for treatment of neurodegenerative diseases or diseases caused by abnormality of RNA-binding proteins and application thereof, especially the application in the treatment of amyotrophic lateral sclerosis (ALS) diseases. The inventors has discovered that the pharmaceutical composition of the present invention such as Olaparib can significantly enhance the dynamics of stress granules containing RNA-binding proteins such as hnRNP A1 (heterogeneous nuclear ribonucleoprotein Al) and TDP-43 (Transactivation response element DNA-binding protein 43), mainly by inhibiting the assembly of stress granules and/or promoting the disassembly of stress granules; affecting the interaction between RNA-binding proteins such as hnRNP A1 and other poly(ADP-ribosyl)ation proteins; affecting the interaction between RNA-binding proteins such as hnRNP A1 and other PAR binding proteins; affects the subcellular localization and stress response of RNA-binding proteins such as hnRNP A1; affecting the liquid-liquid phase separation and aggregation tendency of RNA-binding proteins such as hnRNPA1; affecting co-phase separation of RNA-binding proteins such as hnRNP A1 and TDP-43 protein; affecting the interaction of intracellular RNA-binding proteins such as hnRNP A1 and TDP-43 protein. In addition, in motor neuron-like NSC-34 cells, the pharmaceutical composition such as Olaparib exerts a significant inhibitory effect on neuronal toxicity induced by RNA-binding proteins such as hnRNP A1 and TDP-43.

At present, PARP inhibitors are mostly used in tumor therapy, and there is no research or report showing that drugs that reduce intracellular level of poly(ADP-ribosyl)ation modification, such as Olaparib, can be used in treatment of neurodegenerative diseases such as ALS. The present invention is the first trial of an FDA-approved PARP small molecule inhibitor Olaparib for tumor therapy in treatment of ALS, and found that the inhibitor significantly inhibited the neurocytotoxicity induced by both hnRNP A1 and TDP-43, so the inhibitor can be used to develop clinical drugs for treatment of ALS and other related diseases.

To solve the technical problems described above, the present invention provides a pharmaceutical composition for treatment of neurodegenerative diseases, the pharmaceutical composition comprising a drug that reduce intracellular level of intracellular poly(ADP-ribosyl)ation, preferably drugs that reduce intracellular level of poly(ADP-ribosyl)ation of RNA-binding proteins.

Preferably, the RNA-binding proteins are hnRNP A1 and/or TDP-43.

Preferably, the neurodegenerative disease is amyotrophic lateral sclerosis (ALS).

In order to solve the technical problems described above, the present invention provides a pharmaceutical composition for treatment of diseases caused by abnormality of RNA-binding proteins, the pharmaceutical composition comprises a drug capable of reducing intracellular level of poly(ADP-ribosyl)ation, preferably drugs that reduce level of intracellular poly(ADP-ribosyl)ation of the RNA-binding proteins; the RNA-binding proteins are preferably hnRNP A1 and/or TDP-43.

Preferably, the disease caused by abnormality of the RNA-binding protein is amyotrophic lateral sclerosis (ALS).

Preferably, the treatment of diseases caused by abnormality of RNA-binding proteins comprises one or more of the following:

(a) diseases caused by abnormal intracellular aggregation resulting from abnormal post-translational modification of RNA-binding protein;

(b) diseases caused by abnormal subcellular localization of RNA-binding protein;

(c) diseases caused by abnormality of formation and/or regulation of stress granules in which RNA-binding proteins are involved.

Preferably, the treatment of diseases caused by abnormality of RNA-binding proteins comprises one or more of the following:

(a) diseases caused by abnormality of RNA-binding protein binding and/or regulating processing, shearing, transportation and/or translation of RNA;

(b) diseases caused by interaction between the RNA-binding protein such as hnRNP A1 and other protein like TDP-43 protein and/or subcellular localization of the RNA-binding protein such as hnRNP A1, which is affected by covalent poly(ADP-ribosyl)ation of RNA-binding proteins such as hnRNP A1;

(c) diseases caused by interaction between the RNA-binding proteins such as hnRNP Al and other proteins such as TDP-43 protein, which is affected by non-covalent binding of the RNA-binding proteins such as hnRNP A1 to PAR;

(d) diseases caused by transport of PAR to stress granules under cellular stress conditions, which is affected by non-covalent binding of the RNA-binding proteins to PAR;

(e) diseases caused by destruction of cell homeostasis due to change of solubility of cellular PAR, which is affected by non-covalent binding of the RNA-binding proteins to PAR.

Preferably, the drug capable of reducing intracellular level of poly(ADP-ribosyl)ation in the present invention is a drug capable of increasing expression level of PARG and/or reducing expression level of PARP;

More preferably, the drug capable of increasing expression level of PARG is a PARG agonist;

More preferably, the drug capable of reducing expression level of PARP is a PARP inhibitor; the PARP inhibitor is preferably Olaparib.

In order to solve the technical problem described above, the present invention provides a method for treating the neurodegenerative diseases described above or diseases caused by abnormality of the RNA-binding protein described above, wherein the method comprises treatment using the pharmaceutical composition described above.

Preferably, the pharmaceutical composition provides treatment through one or more of the following mechanisms:

(a) Affecting hnRNP A1 and/or TDP-43 to form stress granules, preferably inhibiting assembly of stress granules and/or promoting disassembly of stress granules;

(b) Affecting interaction between hnRNP A1 and other poly(ADP-ribosyl)ation proteins;

(c) Affecting interaction between hnRNP A1 and other PAR binding proteins;

(d) Affecting subcellular localization and/or stress response of hnRNP A1;

(e) Affecting liquid-liquid phase separation and/or aggregation tendency of hnRNP A1;

(f) Affecting co-phase separation of hnRNP A1 and TDP-43 protein;

(g) Affecting intracellular interaction between hnRNP A1 and TDP-43 protein;

(h) Inhibiting neurocytotoxicity caused by hnRNP A1 and/or TDP-43.

Preferably, the pharmaceutical composition can ultimately inhibit neurodegeneration caused by hnRNP A1 and/or TDP-43. The neurodegeneration is preferably mainly manifested as: 1) inhibition of toxicity in motor neuron like NSC-34 cells, and/or 2) alleviation of phenotype such as neuron degeneration, declined motor ability and shortened life span in Drosophila model of ALS.

In order to solve the technical problems described above, the present invention provides a use of the pharmaceutical composition described above in treatment of the neurodegenerative diseases or diseases described above caused by abnormality of the RNA-binding protein described above.

In order to solve the technical problems described above, the present invention provides a use of the pharmaceutical composition described above in preparing medicament for treatment of the neurodegenerative diseases or diseases described above caused by abnormality of the RNA-binding proteins described above.

On the basis of conforming to common knowledge in the field, the preferred conditions described above can be combined arbitrarily to obtain preferred embodiments of the present invention.

The reagents and raw materials used in the present invention are all commercially available.

The positive and progressive effect of the present invention is as follows: the pharmaceutical composition of the present invention such as Olaparib can significantly enhance the dynamics of stress granules containing RNA-binding proteins such as hnRNP A1 and TDP-43, mainly by inhibiting the assembly of stress granules and/or promoting the disassembly of stress granules; affecting the interaction between RNA-binding proteins such as hnRNP A1 and other poly(ADP-ribosyl)ation proteins; affecting the interaction between RNA-binding proteins such as hnRNP A1 and other PAR binding proteins; affecting the subcellular localization and stress response of RNA-binding proteins such as hnRNP A1; affecting the liquid-liquid phase separation and aggregation tendency of RNA-binding proteins such as hnRNP A1; affecting co-phase separation of RNA-binding proteins such as hnRNP A1 and TDP-43 protein; affecting the interaction of intracellular RNA-binding proteins such as hnRNP A1 and TDP-43 protein. In addition, in motor neuron-like NSC-34 cells, the pharmaceutical composition such as Olaparib exerts a significant inhibitory effect on neuronal toxicity induced by RNA-binding proteins such as hnRNP A1 and TDP-43.

### BRIEF DESCRIPTION OF THE DRAWINGS

FIG. 1 shows that PARylation level affects the dynamic process of assembly-disassembly of stress granules containing hnRNP A1 protein in Example 1.

FIG. 2 shows that PARylation level affects the dynamic process of assembly-disassembly of stress granules containing TDP-43 protein in Example 2.

FIG. 3 shows that the hnRNP A1 protein can not only undergo PARylation, but also bind to PAR in Example 3.

FIG. 4 shows that PARylation or the binding of PAR can affect the subcellular localization and stress response of hnRNP A1 in Example 4.

FIG. 5 shows that PARylation can regulate the interaction between hnRNP A1 and TDP-43 in Example 5.

FIG. 6 shows that PAR can promote the liquid-liquid phase separation of hnRNP A1 in vitro in Example 6.

FIG. 7 shows the ability of hnRNPA1 to undergo co-phase separation with TDP-43, while PAR can facilitate this process in Example 7.

FIG. 8 shows that overexpression of hnRNP A1 or TDP-43 in motor neuron-like NSC-34 cells causes cytotoxity in Example 8.

FIG. 9 shows that PARylation level can regulate the cytotoxicity caused by hnRNP

A1 and TDP-43 in Example 9.

FIG. 10 shows that downregulation of PARP in the Drosophila model of ALS attenuates the neurodegeneration caused by TDP-43 in Example 10.

### DETAILED DESCRIPTION OF THE EMBODIMENT

The present invention will be further illustrated by way of examples below, but the present invention is not thereby limited to the scope of the described embodiments. Experimental methods for which specific conditions are not indicated in the following examples are selected according to conventional methods and conditions, or according to the product specification.

TABLE 1
Sequence enoding human hnRNP A1 protein (SEQ ID
NO: 1):
ATGTCTAAGTCAGAGTCTCCTAAAGAGCCCGAACAGCTGAGGAAGCTCT
TCATTGGAGGGTTGAGCTTTGAAACAACTGATGAGAGCCTGAGGAGCCA
TTTTGAGCAATGGGGAACGCTCACGGACTGTGTGGTAATGAGAGATCCA
AACACCAAGCGCTCCAGGGGCTTTGGGTTTGTCACATATGCCACTGTGG
AGGAGGTGGATGCAGCTATGAATGCAAGGCCACACAAGGTGGATGGAAG
AGTTGTGGAACCAAAGAGAGCTGTCTCCAGAGAAGATTCTCAAAGACCA
GGTGCCCACTTAACTGTGAAAAAGATATTTGTTGGTGGCATTAAAGAAG
ACACTGAAGAACATCACCTAAGAGATTATTTTGAACAGTATGGAAAAAT
TGAAGTGATTGAAATCATGACTGACCGAGGCAGTGGCAAGAAAAGGGGC
TTTGCCTTTGTAACCTTTGACGACCATGACTCCGTGGATAAGATTGTCA
TTCAGAAATACCATACTGTGAATGGCCACAACTGTGAAGTTAGAAAAGC
CCTGTCAAAGCAAGAGATGGCTAGTGCTTCATCCAGCCAAAGAGGTCGA
AGTGGTTCTGGAAACTTTGGTGGTGGTCGTGGAGGTGGTTTCGGTGGGA
ATGACAACTTCGGTCGTGGAGGAAACTTCAGTGGTCGTGGTGGCTTTGG
TGGCAGCCGTGGTGGTGGTGGATATGGTGGCAGTGGGGATGGCTATAAT
GGATTTGGTAATGATGGAAGCAATTTTGGAGGTGGTGGAAGCTACAATG
ATTTTGGGAATTACAACAATCAGTCTTCAAATTTTGGACCCATGAAGGG
AGGAAATTTTGGAGGCAGAAGCTCTGGCCCCTATGGCGGTGGAGGCCAA
TACTTTGCAAAACCACGAAACCAAGGTGGCTATGGCGGTTCCAGCAGCA
GCAGTAGCTATGGCAGTGGCAGAAGATTTTAA
Sequence enoding human TDP-43 protein (SEQ ID
NO: 2):
ATGTCTGAATATATTCGGGTAACCGAAGATGAGAACGATGAGCCCATTG
AAATACCATCGGAAGACGATGGGACGGTGCTGCTCTCCACGGTTACAGC
CCAGTTTCCAGGGGCGTGTGGGCTTCGCTACAGGAATCCAGTGTCTCAG
TGTATGAGAGGTGTCCGGCTGGTAGAAGGAATTCTGCATGCCCCAGATG
CTGGCTGGGGAAATCTGGTGTATGTTGTCAACTATCCAAAAGATAACAA
AAGAAAAATGGATGAGACAGATGCTTCATCAGCAGTGAAAGTGAAAAGA
GCAGTCCAGAAAACATCCGATTTAATAGTGTTGGGTCTCCCATGGAAAA
CAACCGAACAGGACCTGAAAGAGTATTTTAGTACCTTTGGAGAAGTTCT
TATGGTGCAGGTCAAGAAAGATCTTAAGACTGGTCATTCAAAGGGGTTT
GGCTTTGTTCGTTTTACGGAATATGAAACACAAGTGAAAGTAATGTCAC
AGCGACATATGATAGATGGACGATGGTGTGACTGCAAACTTCCTAATTC
TAAGCAAAGCCAAGATGAGCCTTTGAGAAGCAGAAAAGTGTTTGTGGGG
CGCTGTACAGAGGACATGACTGAGGATGAGCTGCGGGAGTTCTTCTCTC
AGTACGGGGATGTGATGGATGTCTTCATCCCCAAGCCATTCAGGGCCTT
TGCCTTTGTTACATTTGCAGATGATCAGATTGCGCAGTCTCTTTGTGGA
GAGGACTTGATCATTAAAGGAATCAGCGTTCATATATCCAATGCCGAAC
CTAAGCACAATAGCAATAGACAGTTAGAAAGAAGTGGAAGATTTGGTGG
TAATCCAGGTGGCTTTGGGAATCAGGGTGGATTTGGTAATAGCAGAGGG
GGTGGAGCTGGTTTGGGAAACAATCAAGGTAGTAATATGGGTGGTGGGA
TGAACTTTGGTGCGTTCAGCATTAATCCAGCCATGATGGCTGCCGCCCA
GGCAGCACTACAGAGCAGTTGGGGTATGATGGGCATGTTAGCCAGCCAG
CAGAACCAGTCAGGCCCATCGGGTAATAACCAAAACCAAGGCAACATGC
AGAGGGAGCCAAACCAGGCCTTCGGTTCTGGAAATAACTCTTATAGTGG
CTCTAATTCTGGTGCAGCAATTGGTTGGGGATCAGCATCCAATGCAGGG
TCGGGCAGTGGTTTTAATGGAGGCTTTGGCTCAAGCATGGATTCTAAGT
CTTCTGGCTGGGGAATGTAG

### Example 1. Effect of PARylation Level on the Dynamic Process of Assembly-Disassembly of Stress Granules Containing hnRNP A1 Protein in Example 1

### 1.1 Experimental Steps

1.1.1 Plasmid Construction

The expression plasmid used in this example is pCAG-hnRNP A1-Flag, and the plasmid was constructed as follows: human hnRNP A1 (Sequence enoding the protein was shown in SEQ ID NO: 1 in Table 1) was fished by PCR from cDNA of HeLa cells (Gene ID: 3178) and inserted into the pCAG plasmid by homologous recombination using ClonExpress™ II One Step Cloning Kit (Vazyme) (this plasmid was constructed by Chen et al. For details, see Chen, Y., Wang, Y., Erturk, A., Kallop, D., Jiang, Z., Weimer, R M, Kaminker, J., and Sheng, M. (2014). Activity-induced Nr4a1 regulates spine density and distribution pattern of excitatory synapses in pyramidal neurons. Neuron 83, 431-443), with EcoRI and XhoI as insertion sites. The Flag tag was added to the primer, and the primer sequences are as follows:

The forward primer of hnRNP A1:
(SEQ ID NO: 3)
5′-CATCATTTTGGCAAAGAATTCCACCATGTCTAAGTCAGAGTCTCCT
AAAGAG-3′
The reverse primer of hnRNP A1:
(SEQ ID NO: 4)
5′-GCTCCCCGGGGGTACCTCGAGCTACTTGTCATCGTCGTCCTTGTAG
AAATCTTCTGCCACTGCCATAGC-3

1.1.2 Cell Culture

HeLa cells were cultivated in Dulbecco's modified Eagle's medium (DMEM) (sigma) containing 10% fetal bovine serum (FBS) (Biowest). Cells were purchased from American Type Culture Collection (ATCC) and grown at 37° C., 5% carbon dioxide. In the present invention, unless otherwise specified, cell were seeded 12-18 hours in advance in 24-well plates (Corning) containing glass slides (Thermo) at a density of 2×10⁵/ml experiments if the cells were used for subsequent immunofluorescence assays, cells were seeded 12-18 hours in advance in 12-well or 6-well plates at a density of 5×10⁵/ml if the cells were used for subsequent WB detection assays.

1.1.3 Cell Processing

Plasmid transfection: The plasmid pCAG-hnRNP A1-Flag constructed in 1.1.1 was transfected with PolyJet™ reagent (SignaGen) (the amount of transfection plasmid is 0.8 μg/ml, and the ratio of transfection reagent to transfection plasmid is 1:2 (mass volume percentage, for example, 1 mg: 2 ml)). Cells were seeded 12-18 hours in advance and transfection was started when the confluence of cell reached about 70%. About 6 hours after transfection, culture medium were changed to ensure growth status of cells. After the exogenous protein was expressed for 24-48 h, the next step of the experiment can be carried out.

Drug treatment: in order to inhibit the level of intracellular PARylation, 20 μM PARP inhibitor Olaparib (Selleck) was added 3 hours before harvesting the plasmid-transfected samples mentioned above, and the control group was treated with an equal volume of DMSO.

RNA interference: in order to increase the level of intracellular PARylation, Lipofectamine™ RNAiMAX Transfection Reagent (Invitrogen) was used to transfect small interfering RNA (Genepharma) while transfecting the plasmids described above. After incubating for 23-48 hours, samples were subsequently collected. The small interfering RNA (siRNA) sequence used in the experiment is as follows:

si-Ctrl: 5′-UUCUCCGAACGUGUCACGUTT-3′ (SEQ ID NO: 5)

si-hPARG: 5′-GCGGUGAAGUUAGAUUACATT-3′ (SEQ ID NO: 6)

1.1.4 Induction and Recovery of Stress Granules

In order to induce the production of stress granules, the HeLa cells collected in 1.1.3 were treated with 100 μM sodium arsenite (Sigma) for 0, 10 and 30 minutes, respectively.

For the stimulation removal experiment, the HeLa cells collected in 1.1.3 were first stimulated with 100 μM sodium arsenite for 30 minutes, rinsed with PBS (Sangon Biotech), and incubated with fresh medium for 0, 30 and 60 minutes.

1.1.5 Immunofluorescence

Cells harvested after treatment in 1.1.4 above were washed once with PBS, fixed with 4% paraformaldehyde (Sangon Biotech) for 15 min, permeabilized with 0.5% Triton-X100 (Sigma) solution for 10 min, and finally incubated with PBST solution containing 3% goat serum (Sigma) (0.1% Tween 20 (Sigma) in PBS) for 1 h at room temperature and incubated overnight at 4° C. with antibodies (Rabbit anti-TIAR (CST), Mouse anti-Flag (Sigma)). The cells were washed with PBST three times at room temperature and incubated for 1 hour at room temperature. After washing three times with PBST at room temperature, fluorescent secondary antibodies (goat anti-mouse-Alexa Fluor 488 (Thermo), goat anti-rabbit-Alexa Fluor 568 (Thermo)) were added for labeling, and then Vectashield Antifade Mounting Medium with DAPI (Vector Laboratories) was used to seal the slices. The signals were observed and acquired under a 100× oil microscope on a Leica TCS SP8 confocal microscope.

1.1.6 Immunoblotting (Western Blot, WB)

Protein extraction: in order to obtain the total intracellular protein of the cells harvested after the treatment in 1.1.4 above, the cells after the corresponding treatment were lysed with 2% (w/v, the same below) SDS cell lysate (the composition of the cell lysate is as follows: 50 mM Tris pH 6.8, 2% SDS, 1% (w/v) β-mercaptoethanol, 12.5% (w/v) glycerol, 0.04% (w/v) bromophenol blue, protease inhibitor (Roche), 20 μM Olaparib (Selleck) and 8 μM ADP-HPD (Millopore)). After the lysed product was centrifuged at high speed at 4° C., the supernatant was added to 4×LDS sample buffer (Invitrogen) and boiled at 95° C.

Immunoblotting assay: the above boiled proteins were separated by 10% Bis-Tris SDS-PAGE (Invitrogen), then transferred to 0.22 μm PVDF membrane (Millipore), sealed with 5% skimmed milk powder, and added with corresponding antibodies (anti-pan-ADP-ribose binding reagent (Millipore), mouse anti-Tubulin (MLB), rabbit anti-PRAG (CST), mouse anti-GAPDH (Proteintech)) for immunoblotting assay. Membranes incubated with primary antibodies were then incubated with the corresponding secondary antibodies coupled with HRP (goat anti-mouse (Sigma), goat anti-rabbit (sigma)), and corresponding protein signals were detected in Amersham Imager 600 (GE Healthcare) using ECL luminescent solution (Tanon).

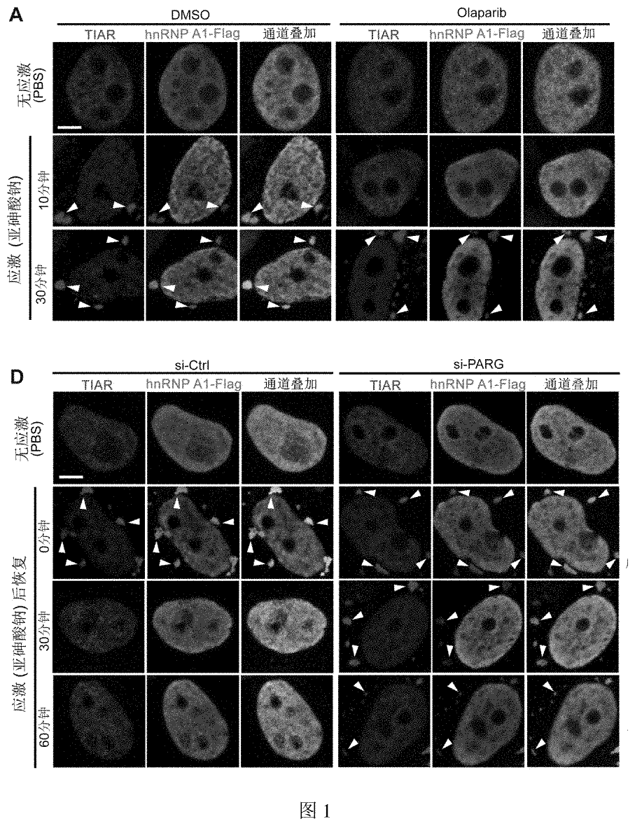

### 1.2. Experimental Results (as Shown in FIG. 1)

(A-C) Inhibition of PARPase activity can hinder the assembly of stress granules: (A) The figure shows representative immunofluorescence images of individual HeLa cells from the experiment. The signal of first channel (red) represents the TIAR protein, which serves as a marker for stress granules; the signal of second channel (green) represents the exogenously expressed hnRNP A1 protein. In the control group (DMSO pretreatment for 3 h), intracellular stress granules appeared gradually with stimulation of sodium arsenite; at the same time, hnRNP Al protein translocated from nucleus to cytosol and formed granules co-localized with red signal of the stress granules. In the experimental group (3 h pretreatment with Olaparib, an inhibitor of PARP), the formation of intracellular stress granules as well as stress granules containing hnRNP Al were delayed. Arrows in the figure indicate hnRNP A1 co-localized with the stress granules. (B) The figure shows the statistics of the percentage (%) of the cells showing stress granules and stress granules containing hnRNP A1 protein in the control (DMSO) and experimental (Olaparib) groups of the experiment shown in (A) at different time points after administration of sodium arsenite stimulation. (C) The figure shows that the PARP inhibitor Olaparib can significantly reduce intracellular level of PARylation which was verified using Western Blot (WB) assay. Wherein, GAPDH (Proteintech) was used as an internal reference to demonstrate the consistent protein loading of the two groups of samples. The results of this experiment show that reducing cellular PARylation levels with PARP inhibitor Olaparib significantly slowed down the formation of stress granules.

(D-F) Reducing intracellular level of PARG enzyme expression delays the process of disassembly of stress granules: (D) sodium arsenite stimulation was removed from cells (drug was replaced with normal cell culture medium), stress granules in control cells (si-Ctrl) gradually disappear with elution time; The dispersion and disappearance of stress granules and hnRNP A1-containing stress granules lagged significantly in experimental group cells (si-hPARG, siRNA that knocks down PARG was added). (E) The figure shows the statistics of the percentage (%) of cells showing stress granules and cells showing stress granules containing hnRNP A1 protein in the control (si-Ctrl) and experimental (si-hPARG) groups of the experiment shown in figure (D) at different time points after removing sodium arsenite stimulation. (F) The figure shows the effect of the knockdown of hPARG protein expression levels using siRNA and increasing intracellular level of PARylaiton detected by WB assay. Tubulin (MBL International Corporation) was used as an internal reference to demonstrate the consistent protein loading of the two groups of samples. The results of this experiment suggest that decreasing intracellular expression of PARG hydrolase and thus increasing the level of intracellular PARylation can significantly delay the disassembly of stress granules in which hnRNP A1 is involved.

All experiments in FIG. 1 were repeated three times, and approximately 100 cells were counted in each group. The significance of statistical differences was determined by testing p value using unpaired, two-tailed Student's t-test. wherein ns represents no significant difference between the two groups; * is p<0.05, ** is p<0.01. The same below.

### Example 2. Effect of PARylation Levels on the Dynamic Process of Assembly-Disassembly of Stress Granules Containing TDP-43 Protein

### 2.1 Experimental Steps

2.1.1 Plasmid Construction

The expression plasmid used in this example is pCAG-TDP-43-HA, and the plasmid was constructed as follows: human TDP-43 (coding sequence for the protein is shown in SEQ ID NO: 2 in Table 1) was amplified from cDNA of HeLa cells by PCR (Gene ID: 23435), and then subcloned into the pCAG plasmid by homologous recombination in insertion sites of EcoRI and XhoI using ClonExpress™ II One Step Cloning Kit (Vazyme). HA tag was added to the primer, and the primer sequences are as follows:

Forward primer of TDP-43:
(SEQ ID NO: 7)
5′-CATCATTTTGGCAAAGAATTCCACCATGTCTGAATATATTCGGGTA
AC-3′
Reverse primer of TDP-43:
(SEQ ID NO: 8)
5′-GCTCCCCGGGGGTACCTCGAGTTAAGCGTAGTCTGGGACGTCGTAT
GGGTACATTCCCCAGCCAGAAGACTT-3

2.1.2 the Rest of the Experimental Reagents and Experimental Procedures Used Herein are the Same as Those Used in Example 1.

Wherein, the primary antibodies used in immunofluorescence were Rabbit anti-TIAR (CST) and Mouse anti-HA (Proteintech). Secondary antibodies were goat anti-mouse-Alexa Fluor 488 (Thermo) and goat anti-rabbit-Alexa Fluor 568 (Thermo).

### 2.2. Experimental Results (as Shown in FIG. 2)

(A-B) Inhibition of PARPase activity can hinder the assembly of stress granules: (A) The figure shows representative immunofluorescence images of individual HeLa cells in the experiment. The basic logic of this embodiment is the same as shown in FIG. 1 (A), except that the detection target was changed to another important ALS-related RNA-binding protein, TDP-43. The signal of first channel (red) represents the TIAR protein, which is a marker for stress granules; the signal of second channel (green) represents the exogenously expressed TDP-43 protein. The results show that pretreatment with Olaparib, an inhibitor of PARP, delayed the formation of intracellular stress granules as well as stress granules containing TDP-43. Arrows in the figure indicate TDP-43 co-localized with the stress granules. (B) The figure shows the statistics of the percentage (%) of the cells showing stress granules and cells showing stress granules TDP-43 protein in the control (DMSO) and experimental (Olaparib) groups of the experiment shown in (A), at different time points after administration of sodium arsenite stimulation. The results of this experiment show that reducing cellular PARylation levels with PARP inhibitor Olaparib significantly slowed down the formation of stress granules in which TDP-43 is involved.

(C-D) Reducing intracellular level of PARG enzyme expression slows down the process of disassembly of stress granules: (C) The basic logic of this figure is the same as that in FIG. (1D), which shows that knocking down PARG using siRNA leads to a significant slowdown in the rate of disassembly and disappearance of intracellular stress granules and TDP-43-containing stress granules after removing stimulation. (D) The figure shows the statistics of the percentage (%) of the cells showing stress granules and cells showing stress granules containing TDP-43 protein in the control (si-Ctrl) and experimental (si-hPARG) groups of the experiment shown in (C) at different time points after removing sodium arsenite stimulation. The results of this experiment suggest that decreasing intracellular expression of PARG hydrolase and thus increasing the level of intracellular PARylation can significantly delay the disassembly of stress granules in which TDP-43 is involved.

All experiments in FIG. 2 were performed in triplicate, and approximately 100 cells were counted in each group. The determination of the statistical difference is the same as that in Example 1.

From the results of Examples 1 and 2, it can be seen that the level of PARylation affects the dynamic process of assembly-disassembly of stress granules involving hnRNPA1 and TDP-43. And since a close relationship between stress granules and neurodegenerative diseases such as ALS has been reported in the prior art (Li et al., 2013), a correlation between PARylation and ALS is suggested.

### Example 3. hnRNPA1 Protein can Both be PARylated and Bind to PAR

### 3.1 Experimental Steps

3.1.1 Plasmid Construction

The expression plasmids used in this example including pCAG-hnRNPA1-K298A-Flag and pCAG-hnRNP A1-PBMmut-Flag plasmids (PBM is short for PAR-binding motif) were obtained using the pCAG-hnRNP A1-Flag from Example 1 as a template and single-site mutagenesis PCR using Fast Mutagenesis Kit II (Vazyme), and the primer sequences used are as follows.

The primers corresponding to pCAG-hnRNP A1-K298A-Flag:

Upstream primer of hnRNP A1-K298A:
(SEQ ID NO: 9)
5′-CTTTGCAGCACCACGAAACCAAGGTGGCTATGGCGG-3′
Downstream primer of hnRNP A1-K298A:
(SEQ ID NO: 10)
5′-TTCGTGGTGCTGCAAAGTATTGGCCTCCACCGCCATAGG-3

The primers corresponding to pCAG-hnRNP A1-PBMmut-Flag:

Upstream primer of hnRNP A1-PBM^{mut}:
(SEQ ID NO: 11)
5′-GGTGCCCACTTAACTGTGGTTGTTATATTTGTTGGTGGCATTAAAG
AAGACACTGAAGAAC-3
Downstream primer of hnRNP A1-PBM^{mut}:
(SEQ ID NO: 12)
5′-CCACCAACAAATATAACAACCACAGTTAAGTGGGCACCTGGTCTTT
GAGAA-3′

3.1.2 Induction of Intracellular PARylation

The steps of HeLa cell culture and plasmid transfection are as described in Example 1. To increase the level of PARylation in HeLa cells after transfection with the plasmids described in 3.1.1, the cell culture medium was removed before collection, 500 μM hydrogen peroxide (diluted in PBS) was added to the cells followed by incubating for 10 min at 37° C. The stimulated HeLa cells can be used for subsequent experiments.

3.1.3 Protein Extraction

Cells were lysed by adding 2% SDS cell lysate after corresponding treatment as described in 3.1.2 (cell lysate composition is as follows: 50 mM Tris pH 6.8, 2% SDS, 1% β-mercaptoethanol, 12.5% glycerol, 0.04% bromophenol blue, protease inhibitor (Roche), 20 μM Olaparib (Selleck) and 8 μM ADP-HPD (Millopore)). After the lysed product was centrifuged at high speed at 4° C., the supernatant was pipetted to 4×LDS sample buffer (Invitrogen) and boiled at 95° C.

3.1.4 Immunoprecipitation (IP)

Cells treated according to 3.1.2 were lysed with the following lysis buffer: 25 mM Tris-HCl pH 7.4, 150 mM NaCl, 1% NP-40, 1 mM EDTA, 5% glycerol, protease inhibitor (Roche), 20 μM Olaparib (Selleck) and 8 μM ADP-HPD (Millopore). For enrichment of TDP-43 with HA tag, the lysate was added with HA antibody (Mouse anti-HA (Proteintech)) for overnight incubation at 4° C. followed by enrichment of primary antibody-sample complexes with Dynabeads® Protein G beads (Novex) for 2 hours at room temperature; for Flag-tagged hnRNP Al, the lysate was added directly to anti-FLAG® M2 Affinity Gel (Sigma) followed by incubation at 4° C. To ablate the polyribose moiety in vitro, 1μ purified PARG protein (Sigma) was added to a mixture of anti-FLAG® M2 Affinity Gel (Sigma) and cell lysate, and incubated for 1 h at 37° C. After incubation, the product was washed 3-5 times with lysis buffer, and added with 4×LDS sample buffer (Invitrogen) and boiled at 95° C. for subsequent studies.

3.1.5 Immunoblotting (Western Blot, WB)

The proteins boiled according to 3.1.3 were separated by 10% Bis-Tris SDS-PAGE (Invitrogen) and transferred onto 0.22 μm PVDF membrane (Millipore), blocked with 5% skimmed milk powder at room temperature for 1 h, and then the corresponding antibodies were added for immunoblotting detection. Membranes incubated with primary antibodies (Rabbit anti-HA (CST), Mouse anti-Flag (Sigma) and anti-pan-ADP-ribose binding reagent (Millipore)) were then incubated with the corresponding secondary antibodies coupled to HRP (goat anti-HA (Sigma), goat anti-rabbit (Sigma)), followed by detecting the signals of corresponding proteins in Amersham Imager 600 (GE Healthcare) using ECL luminescent solution (Tanon).

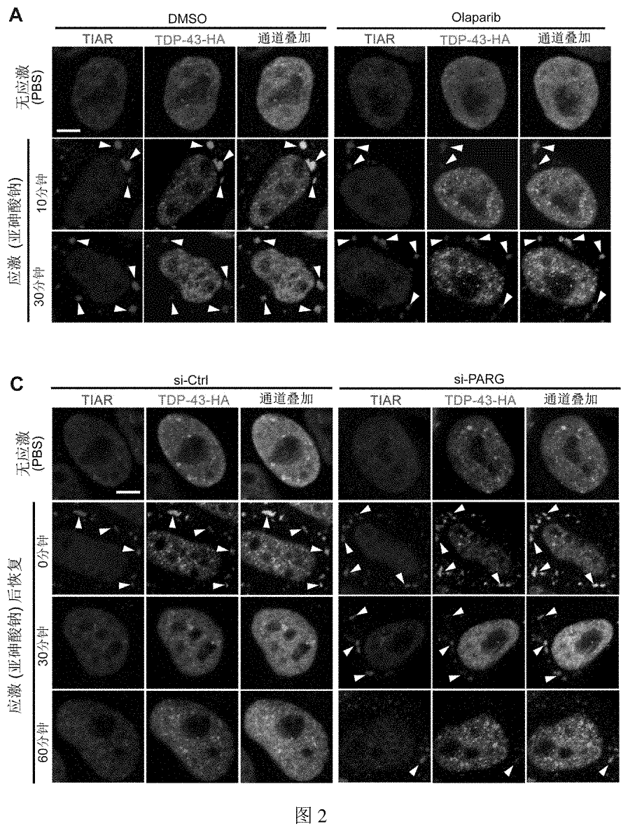

### 3.2. Experimental Results (as Shown in FIG. 3)

(A) The figure shows the capability of TDP-43 being PARylated in HeLa cells, which was determined by WB. Exogenously transfected TDP-43-HA was enriched by immunoprecipitation, and modified TDP-43 was not detected using antibodies for PAR (indicated by arrows). Under the condition of adding hydrogen peroxide to activate intracellular PARPase activity and increase the level of intracellular PARylation, PARylated TDP-43 or other PARylated proteins that interact with TDP-43 was still not detected. The above results suggest that TDP-43 is difficult to be PARylated in vivo. * Represents antibody heavy chain.

(B) The figure shows the capability of A1 being PARylated in HeLa cells, determined by WB. Weak PARylation modification of hnRNP A1 under physiological conditions (indicated by arrows) can be detected by using exogenously transfected hnRNP A1-Flag which was enriched by IP. Further increasing intracellular PARylation levels not only significantly increased the PARylation level of hnRNP A1 itself, but also more PARylated proteins that interact with hnRNP A1 can be detected (indicated by #).

(C) Upon the stimulation of hydrogen peroxide, intracellular hnRNP A1 itself had a higher level of PARylation modification, and there are more PARylated proteins that interact with it. However, if PARG protein was added to the IP system for 1 h, both the PARylation level of hnRNP A1 itself and the PARylated proteins that interact with it would be significantly reduced, indicating that the signal detected in WB was indeed caused by PARylation.

(D) Schematic diagram of the functional domain of hnRNP A1 protein. Human hnRNP Al contains two RNA recognition motifs (RRM) and one glycine-rich domain (GRD) located at the C-terminus. The specific sequence at the top of the schematic diagram is the potential PARylation site Lys298 of hnRNP A1 (marked in bold). In this example, this site was mutated to Ala (marked by underline), and subsequently is abbreviated as K298A; the specific sequence at the bottom of the schematic is a potential PAR-binding motif (PBM) between two RRMs, the amino acid sequence of this domain is conservative (PBM consensus), where “h” stands for hydrophobic amino acids and “b” stands for basic amino acids. In this example, the three key amino acids (marked in bold) of the domain were mutated (marked by underline), and recorded as PBM^{mut} in subsequent experiments.

(E) The figure shows the difference in PARylation and PAR binding ability of WT, K298A and PBM^{mut} hnRNP A1 in cells detected by WB. Compared with WT, the K298A mutation significantly reduced the level of hnRNP A1's own PARylation (indicated by arrow), but the ability to bind to PAR did not change significantly (indicated by #); while PBM^{mut} destroyed the ability of hnRNP A1 to bind to PAR, but its own PARylation level rised to a certain extent. Tubulin was used as an internal reference to demonstrate the consistent protein loading of the three groups of samples. The above results suggest that the binding of hnRNP A1 to PAR via PBM may inhibit excessive PARylation at the K298 position.

It can be seen from this example that hnRNP A1 itself can both be poly(ADP-ribosyl)ated and interact with other PARylated proteins by binding to PAR.

### Example 4. Effect of PARylation or PAR Binding on the Subcellular Localization

And Stress Response of hnRNP A1

### 4.1 Experimental Steps

Experimental steps are the same as those in Example 1-3.

### 4.2. Experimental Results (as Shown in FIG. 4)

(A) Subcellular localization of WT, K298A, PBM^{mut} three hnRNP A1 proteins in HeLa cells and their response to cell pressure were detected by immunostaining. In this figure, the signal of first channel (red) represents the TIAR protein, which serves as a marker for stress granules; the signal of second channel (green) represents the exogenously expressed hnRNP A1 protein. In the absence of external stimulation (PBS), WT and K298A were almost all located in the nucleus. Although PBM^{mut} was mainly located in the nucleus, a certain amount of protein forms abnormal cytoplasmic foci (arrow with tail). Under the stimulation of sodium arsenite, WT transferred from the nucleus to the cytoplasm and co-localized with the stress granules (arrow). Although the cells transfected with K298A can form stress granules normally, the mutated hnRNP A1 can hardly be exported from the nucleus (*). In the case of PBM^{mut} stimulation, although a small amount of protein can normally enter the stress granules (arrow), there were still a large number of abnormal cytoplasmic foci formed, which cannot co-localize with the stress granules (arrow with tail).

Figs. (B-E) shows statistics of the percentage of cells that can normally form stress granules (B) in figure (A), the percentage of cells that form stress granules containing hnRNP Al (C), the percentage of cells that form abnormal cytoplasmic foci (D), and the number of abnormal cytoplasmic foci formed in each cell (E). The above results indicate that the PARylation at K298 may be the nuclear exporting signal of hnRNP A1, and the binding of PAR through PBM can help hnRNP A1 translocate to stress granules under sodium arsenite stimulation.

All experiments in FIG. 4 were performed in triplicate, and approximately 300 cells were counted in each group. The unpaired, two-tailed Student's t-test method was used to determine p value of the same genotype before and after sodium arsenite stimulation; differences in stimulus responses between genotypes were compared by two-way ANOVA, where ns represents no significant difference between the two groups; * is p<0.05, ** is p<0.01, and *** is p<0.001.

The results of Examples 3 and 4 illustrate that the covalent PARylation of hnRNP A1 pathogenic protein affected its subcellular localization, whereas non-covalent binding of PAR would affect the transport of hnRNP A1 protein to stress granules under cellular stress conditions. The ALS caused by hnRNP A1 is closely related to its abnormal subcellular localization and stress response (see “Background arts”). Therefore, this part of data provides the cell regulation mechanism and experimental basis for improving the level of PARylation and developing PARP inhibitors for the treatment of ALS.

### Example 5. Regulation of PARylation on the Interaction Between hnRNP A1 and TDP-43

### 5.1 Experimental Steps

Experimental steps are the same as those in Example 3.

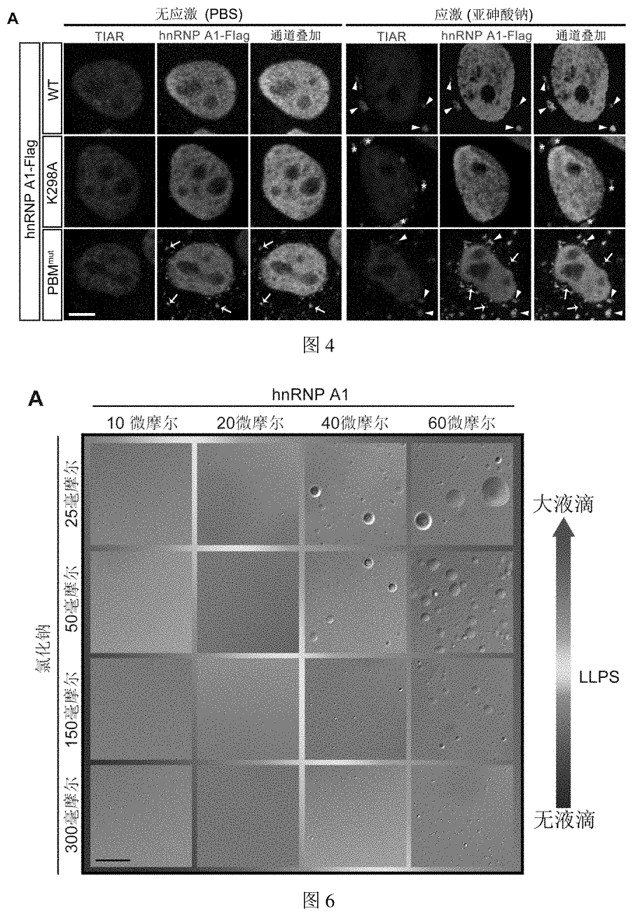

### 5.2. Experimental Results (as Shown in FIG. 5)

(A) The interaction between hnRNP A1 and TDP-43 is reduced by hydrolysis of PAR: it was detected by immunoprecipitation assay that hnRNP A1 is able to interact with endogenous TDP-43; the poly ADP ribose moiety was further hydrolyzed by addition of PARG to the IP system, and the amount of endogenous TDP-43 protein that can be co-precipitated is significantly reduced.

(B) Statistics of the relative expression of endogenous TDP-43 protein obtained by immunoprecipitation in figure (A).

(C) Increasing PARylation levels enhanced the interaction between hnRNP A1 and TDP-43: the intracellular PARylation level enhanced by the dual treatment of hydrogen peroxide stimulation and si-PARG can increase the amount of TDP-43 protein obtained by immunoprecipitation with hnRNP A1.

(D) Statistics of the relative expression of endogenous TDP-43 protein obtained by immunoprecipitation in figure (C);

All experiments in FIG. 5 were performed in triplicate-quintuplicate. The significance of statistical difference was determined by unpaired, two-tailed Student's t-test method to test the p value, *** is p<0.001.

### Example 6. PAR Promotes the Liquid-Liquid Phase Separation (LLPS) of hnRNP A1 In Vitro

### 6.1 Experimental Steps

6.1.1 Protein Expression and Purification

pET9d-hnRNP A1 (Addgene) was used for hnRNP A1 protein expression in E. coli BL21 (DE3) pLysS (TranGeneBiotech). After 0.4 mM IPTG (SangonBiotech) induction at 25° C. for 15 h, the obtained bacteria were collected by centrifugation, and lysed by lysis buffer (50 mM Tris-HCl at pH 7.5, 2 mM DTT, 1 mM PMSF, 5% glycerin, and 0.1 mg/mL RNase A). Supernatant was collected by centrifugating the lysate at a high speed at 4° C. and then transferred to a 5 ml SP column in an AKTA (GE Healthcare) machine. The protein binded on the column was eluted with a mixture (9:1) of buffer A (50 mM Tris-HCl pH 7.5, 2 mM DTT and 5% glycerin) and buffer B (buffer A with 1 M NaCl). The eluted protein was purified by Superdex 75 16/600 column (GE Healthcare). The purified protein was dissolved in stock buffer (50 mM Tris-HCl pH 7.5, 500 mM NaCl and 2 mM DTT) for subsequent experiments.

6.1.2 In Vitro LLPS Experiment

The purified hnRNP A1 protein was mixed with a certain concentration of PAR (Trevigen) under a certain salt concentration. The reaction system was 50 mM Tris-HCl, pH 7.5, 10% (w/v) PEG 3550 (Sigma) and 2 mM DTT. After the mixture was incubated at room temperature for 3 minutes, 5 μl was dropped on a glass slide for observation with an SP8 confocal microscope (Leica).

6.2. Experimental Results (as Shown in FIG. 6)

(A) hnRNP A1 can spontaneously form droplets through LLPS: the picture shows that hnRNP A1 forms droplets through phase separation at a specific protein concentration and salt ion concentration. With the increase of protein concentration and the decrease of salt ion concentration, the diameter and number of hnRNP A1 droplets gradually increased.

(B) Addition of different concentrations of PAR under critical conditions promoted LLPS of hnRNP A1, and the promotion ability gradually increased with increasing PAR concentration.

(C) High concentrations of heparin (Heparin) did not promote LLPS of hnRNP A1.

(D) High concentrations of PAR cannot induce LLPS on their own. The above results indicate that PAR is specific for promoting LLPS of hnRNP A1.

### Example 7. hnRNP A1 can Co-Phase Separate with TDP-43 and PAR can Facilitate this Process

### 7.1 Experimental Steps

7.1.1 Plasmid Acquisition

The plasmid pET9d-hnRNP A1 used for in vitro expression purification was purchased from Addgene. The other two plasmids pET-28a-TDP-43¹⁻²⁷⁴-6×His and pET-28a-TDP-43²⁷⁴⁻⁴¹⁴ 6×His (pET28a plasmid from Addgene) were constructed by essentially the same procedure as in Example 1, with insertion sites BamHI and XhoI. The primers used are as follows:

Primers corresponding to pET-28a-TDP-43¹⁻²⁷⁴-6×HIS.

Upstream primer for TDP-43¹⁻²⁷⁴:
(SEQ ID NO: 13)
5′-ATGGCCATGGAGGCCGAATTCATGTCTGAATATATT-3′
Downstream primer of TDP-43¹⁻²⁷⁴:
(SEQ ID NO: 14)
5′-CATGTCTGGATCCCCGCGGCCGCCTAACTTCTTTCTAACTGTCTA
TT-3′

The primers corresponding to pET-28a-TDP-43274-414-6×His:

Upstream primers for TDP-43²⁷⁴⁻⁴¹⁴:
(SEQ ID NO: 15)
5′-CAGCAAATGGGTCGCGCCACCGGATCCGGAAGATTTGGTGGT-3′
Downstream primers of TDP-43²⁷⁴⁻⁴¹⁴:
(SEQ ID NO: 16)
5′-GTGGTGGTGGTGGTGCTCGAGCATTCCCCAGCCAGA-3

7.1.2 Protein Expression and Purification

TDP-43¹⁻²⁷⁴: The expression plasmid pET-28a-TDP-43¹⁻²⁷⁴-6×His was expressed in BL21 E. coli (DE3) (TranGeneBiotech) after induction by 50 uM IPTG (SangonBiotech) at 19° C. for 16 h. The resulting bacteria were collected by centrifugation, and lysed by lysis buffer (50 mM Tris-HCl, 500 mM NaCl, pH 8.0, 10 mM imidazole, 4 mM β-mercaptoethanol, 1 mM PMSF, and 0.1 mg/mL RNase A). Supernatant was collected by centrifugating the lysate at low temperature and then purified and enriched by Ni column (GE Healthcare). The protein enriched by Ni columns was eluted with eluent (50 mM Tris-HCl, 500 mM NaCl, pH 8.0, 250 mM imidazole and 4 mM β-mercaptoethanol), and then purified with Superdex 200 1616/600 column (GE Healthcare). The purified protein was finally dissolved in a storage buffer (50 mM Tris-HCl pH 7.5, 300 mM NaCl and 2 mM DTT) for subsequent experiments.

TDP-43²⁷⁴⁻⁴¹⁴: This protein was expressed in BL21 E. coli (DE3) (TranGeneBiotech) by adding 50 uM IPTG (SangonBiotech) at 37° C. for inducing the expression of the protein in inclusion bodies. Collected bacteria were lysed with denaturing lysis buffer (50 mM Tris-HCl, pH 8.0 and 6 M guanidine hydrochloride). The lysis product was centrifuged at high speed for 1 h at 4° C. and the supernatant was collected for purification by Ni column. The proteins were eluted with denaturing elution solution (50 mM Tris-HCl at pH 8.0, 6 M guanidine hydrochloride and 50 mM imidazole) and then further purified by HPLC (Agilent) and the resulting product was lyophilized using a freeze-dryer (Thermo Fisher) and then prepared for use.

7.1.3 Fluorescent Labeling of Protein

The purified TDP-43¹⁻²⁷⁴ and hnRNP A1 proteins (obtained in Example 6) were diluted separately into desalting reaction solution (50 mM Tris-HCl, pH 7.5, 500 mM NaCl and 4 mM Tris(2-Carboxyethyl) Phosphine (TCEP) (Invitrogen)), and DTT was removed using a desalting column (GE Healthcare). After desalting, TDP-43¹⁻²⁷⁴ and hnRNP A1 proteins were incubated for 2 h at room temperature with 5 times the volume of AlexaFluor-555 C2-malemide (Invitrogen) or AlexaFluor-647 C2-malemide (Invitrogen), respectively. The eluted protein was purified by Superdex 75 16/600 column (GE Healthcare) for later use.

7.1.4 In Vitro LLPS Experiment

The purified hnRNP A1 and TDP-43 were mixed with corresponding protein concentrations under a certain salt concentration. The reaction system was 50 mM Tris-HCl pH 7.5 and 2 mM DTT. After the mixture was incubated at room temperature for 3 minutes, 5 μl was added dropwise on a glass slide for observation with an SP8 confocal microscope (Leica).

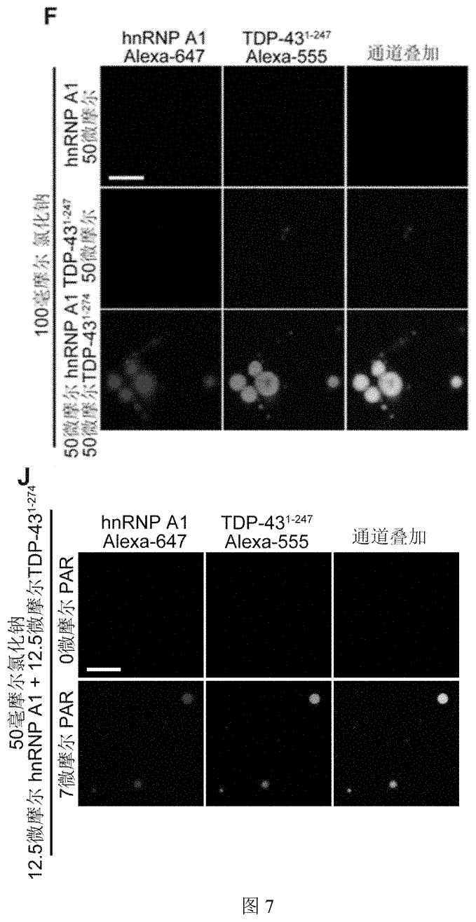

7.2. Experimental Results (as Shown in FIG. 7)

(A) Under the protein and salt ion concentration shown in the figure, hnRNP A1 cannot spontaneously form droplets.

(B-E) The picture shows the phase separation between hnRNP A1 and BSA (B), TDP-43¹⁻²⁷⁴ (C) or TDP-43²⁷⁴⁻⁴¹⁴ (D-E) under specified conditions. (B′-E′) The picture shows the detection of the phase separation ability of BSA (B′), TDP-43¹⁻²⁷⁴ (C′) or TDP-43²⁷⁴⁻⁴¹⁴ (E′) under the same conditions as figure (B-E). The above results show that under the conditions of this embodiment, hnRNP A1 can only co-phase separate with TDP-431-274

(F) Fluorescence-labeled protein (A, C, C′) verified that hnRNP A1 and TDP-43¹⁻²⁷⁴ underwent a phase separation under reaction conditions of figure (black box). The first channel (red) represents hnRNP A1 and the second channel (green) represents TDP-43^{1-274.} Droplets can be formed only when the two are mixed and the two droplets can co-localize well. The above results indicate that the droplets formed after the mixing of hnRNP A1 and TDP-43¹⁻²⁷⁴ were the result of a co-phase separation, rather than a promotion by one party alone to the other.

(G) PAR can promote the co-phase separation of hnRNP A1 and TDP-43¹⁻²⁷⁴, and the degree of promotion was positively correlated with the PAR concentration.

(H) High concentration of PAR cannot promote the co-phase separation of hnRNP A1 and TDP-43²⁷⁴⁻⁴¹⁴

(I) Adding high concentration of PAR to hnRNP A1 or TDP-43¹⁻²⁷⁴ cannot promote their phase separation.

(J) Fluorescence-labeled protein (G) verified that PAR promoted co-phase separation of hnRNP A1 and TDP-431-274 under the specified conditions of figure (black box).

Examples 6 and 7 illustrate that the addition of PAR promoted the co-phase separation of hnRNP A1 and TDP-43, and molecular mechanism mediating the formation of intracellular stress granules is the co-phase separation of proteins in vitro (see “Background arts” for details). The results of this in vitro experiment corroborate with the results of the cellular assays of Example 5, illustrating that increasing PARylation levels promotes interaction between hnRNP Al and TDP-43 and enhances stress granule assembly, which is the basis for elucidating the molecular mechanism of PARP inhibitor to maintain stress granule dynamics by mitigating protein interactions and preventing their progression to the insoluble protein aggregates shown in ALS.

### Example 8. Overexpression of hnRNP A1 or TDP-43 in Motor Neuron-Like NSC-34 Cells Causes Cytotoxicity

### 8.1 Experimental Steps

8.1.1 Cell Culture

293T cells were cultured in DMEM (sigma) containing 10% FBS (Biowest). NSC-34 cells were cultured with RPMI 1640 medium (Gibco, 11875-093) containing 10% FBS. Cells were grown at 37° C. with 5% carbon dioxide. Cells were both purchased from ATCC.

8.1.2 TUNEL Staining

TUNEL staining was performed using TMR red in situ Cell Death Detection Kit (Sigma-Aldrich) according to the instructions in the kit.

8.1.3 Virus Production and Infection

Lentiviral packaging plasmids and the corresponding exogenous hnRNP A1 or TDP-43 expression plasmids were transfected simultaneously in 293T cells with PolyJet™ transfection reagent. After 48 hours of transfection, the cell culture was collected by filtration with a 0.45 μm syringe filter (Millipore) and concentrated with Lenti-X™ Concentrator (Clontech), and the concentrated product was used for subsequent infection of NSC-34 cells.

8.1.4 Cell Viability Assay

NSC-34 cells were seeded one day in advance in 96-well plates (Corning) and the corresponding lentivirus was added for transfection. Cell viability was determined 48-72 h after transfection using Cell Counting Kit-8 (CCK-8) (Dojindo) kit. In brief, 10 μL of CCK-8 solution was added to each well. After incubation at 37° C. for 2.5 h, the absorbance light at 450 nm of the solution was detected with a Synergy2 microplate reader (BioTek Instruments) and cell viability values were calculated according to the instructions.

### 8.2. Experimental Results (as Shown in FIG. 8)

(A-B) Overexpression of hnRNP A1 induces cytotoxicity in NSC-34 cells: it was observed by bright-field microscopy that NSC-34 cells overexpressing hnRNP A1 was abnormal in morphology, and there was significant cell death (A). CCK-8 detected that hnRNP A1 overexpression induces a decrease in cell viability and a further decrease in cell viability with increasing amounts of lentivirus used to overexpress hnRNP A1 (B).

(C-F) Overexpression of TDP-43 induces cytotoxicity in NSC-34: overexpression of TDP-43 induces abnormal NSC-34 cell morphology (C) and dose-dependent decrease in cell viability (D). TUNEL staining assay showed that overexpression of TDP-43 can cause cell death (F). The first channel (green) in the figure represents TDP-43; the second channel (red) is the TUNEL signal, indicating that the cell was dead; the third channel (blue) is DAPI, indicating the nucleus.

All experiments in FIG. 8 were performed in triplicate. The significance of statistical differences was determined by the unpaired, two-tailed Student's t-test method to test the p value, where ns represents no significant difference between the two groups; * is p<0.05 and ** is p<0.01.

### Example 9. PARylation Levels can Regulate Cytotoxicity Induced by hnRNP A1 and TDP-43

### 9.1 Experimental Steps

9.1.1 siRNA Transfection

The siRNA transfection procedure was the same as that in Example 1, and the transfection time was 48-60 h. The corresponding siRNA sequences are as follows.

si-mPARG:
(SEQ ID NO: 17)
5′-GCAGUUUCUUACACCUAUATT-3′
si-mPARP1:
(SEQ ID NO: 18)
5′ -CGACGCUUAUUACUGUACUTT-3′

9.1.2 qPCR Detection

For the extraction of total mRNA from NSC-34 cells, the cells were collected after the corresponding treatment, lysed completely by Trizol (Invitrogen), followed by chloroform extraction and isopropanol precipitation to obtain mRNA, and then DNase (Promega) was added to eliminate the contamination of genomic DNA. 1 μg of mRNA was taken for reverse transcription using High-Capacity cDNA Reverse Transcription Kit (Applied biosystems). The obtained cDNA was mixed with SYBR Green qPCR Master Mix (Bimake) and primers of target gene by a certain ratio and then subjected to qPCR assay (QuantStudio™ 6 Flex Real-Time PCR system (Life Technologies)). The primers used are as follows.

mParg:
Upstream primer:
(SEQ ID NO: 19)
5′-AGCCTCTGACACGCTTACAC-3′;
Downstream primer:
(SEQ ID NO: 20)
5′-CAGTCACACCACCTCCAACA-3
mGAPDH:
Upstream primer:
(SEQ ID NO: 21)
5′-CACCATCTTCCAGGAGCGAG-3′;
Downstream primer:
(SEQ ID NO: 22)
5′-CCTTCTCCATGGTGGTGAAGAC-3′

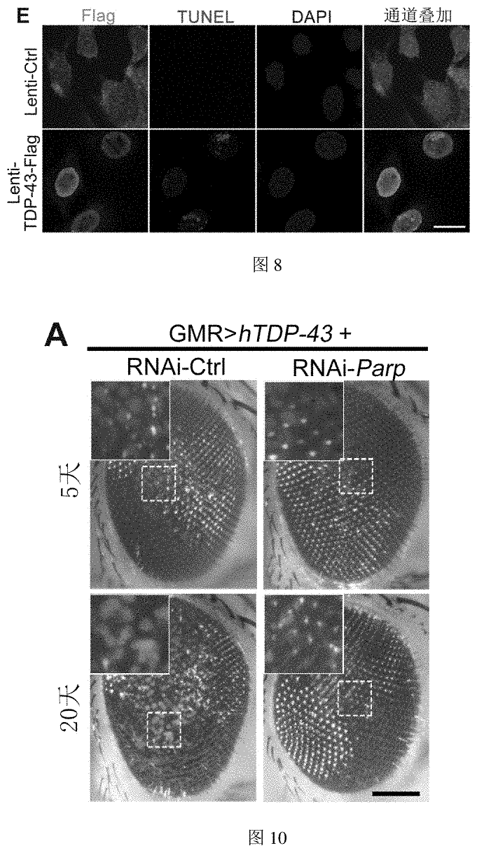

9.2. Experimental Results (as Shown in FIG. 9)

(A-C) Knockdown of PARP1 alleviates cytotoxicity induced by hnRNP A1 or TDP-43. Transfection of NSC-34 cells with hnRNP A1 (A) or TDP-43 (B) resulted in a significant reduction in cell viability, which was alleviated by knockdown of PARP1 (si-PARP1). (C) The figure shows the effect of knocking down PARP1 protein expression level using siRNA and reducing intracellular level of PARylaiton detected by WB assays. GAPDH was used as an internal reference to demonstrate the consistent protein loading of the two groups of samples. The above results suggest that reducing intracellular PARylation levels by genetic means can alleviate the cytotoxicity induced by hnRNP A1 and TDP-43. Meanwhile, this result suggests that PARP1 plays a key role in the regulation of hnRNP A1 and TDP-43 by PARylation.

(D-F) Knockdown of PARP1 alleviates cytotoxicity induced by hnRNP A1 or TDP-43. The increase of the level of overall cellular PARylation by si-PARG and the knock down of the mRNA level of PARG by si-PARG were detected by WB and qPCR, respectively (D). Knockdown of PARG in NSC-34 cells further enhanced hnRNP A1 (E) or TDP-43 (F) induced cytotoxicity. The above results suggest that increasing intracellular PARylation levels by genetic means can enhance the cytotoxicity induced by hnRNP A1 and TDP-43.

(G) The PARP inhibitor Olaparib can alleviate TDP-43-induced cytotoxicity. 5 μM Olaparib can reduce the cytotoxicity induced by overexpression of TDP-43. This result shows that the reduction of the level of intracellular PARylation at the pharmacological level can alleviate the cytotoxicity induced by TDP-43.

All experiments in FIG. 9 were performed in triplicate. The significance of statistical differences was determined by the unpaired, two-tailed Student's t-test method to test the p value, where ns represents no significant difference between the two groups; * is p<0.05, ** is p<0.01, *** is p<0.001.

As described in the background arts, a major feature of ALS is the death and loss of motor neurons. The present invention utilizes a motor neuron cell type NSC-34 as a model to study the effects of cytotoxicity on motor neurons induced by hnRNP A1 and TDP-43. Overexpression of hnRNP A1 or TDP-43 in NSC-34 cells can trigger abnormal effects on cells similar to those produced by these two pathogenic proteins in ALS, such as changes in cell morphology, DNA break during apoptosis (detected by TdT-mediated dUTP Nick-End Labeling (TUNEL) staining) and changes in cell number and viability (detected by the Cell Counting Kit-8 (CCK-8) method). The occurrence of cell death or reduced viability indicates that abnormalities in hnRNP A1 or TDP-43 proteins induce cytotoxicity, which corresponds to ALS that abnormalities in these two proteins may cause motor neuron death and ultimately lead to the development of ALS. In contrast, the results of Examples 8 and 9 show that the reduction of the level or activity of PARP, either by a genetic or pharmacological approach, effectively inhibits the cytoxicity to motor neuron-like NSC-34 cells induced by overexpression of hnRNP A1 and TDP-43. This result provides the most direct and powerful indication that PARP inhibitors are expected to delay motor neuron death in ALS patients.

### Example 10. Down-Regulation of PARP in the ALS Drosophila Model can Attenuate Neurodegeneration Induced by TDP-43

### 10.1. Experimental Procedure

10.1.1 Drosophila Acquisition

The Drosophila used in the present invention are from Bloomington Drosophila Stock Center (BDSC): RNAi-Parp (#57265), elavGS (#43642), RNAi-mCherry (#35785, the RNAi-ctrl flies used in the experiment), GMR-GAL4 (#79573). UAS-TDP-43 fly was obtained by site insertion of target gene into the Drosophila genome with ΦC31 transposase. Correspondingly, mating fly with specific RNAi or UAS flies can reproduce offspring that meet the requirements. All experimental flies were fed in standard corn flour medium at 25° C. and 60% humidity. The Drosophila used in the experiment are all male, and the complete genotypes are as follows:

The complete genotype when used for the detection of Drosophila mRNA levels:

w; UAS-RNAi-mCherry/GMR-GAL4

w; UAS-RNAi-Parp/GMR-GAL4

The complete genotype for the detection of Drosophila optic nerve degeneration and exogenously expressed protein:

w; UAS-hTDP-43/+; UAS-RNAi-mCherry/GMR-GAL4

w; UAS-hTDP-43/+; UAS-RNAi-Parp/GMR-GAL4

The complete genotype when used to test the crawling ability and survivability of Drosophila:

w; UAS-hTDP-43/+; UAS-RNAi-mCherry/elavGS

w; UAS-hTDP-43/+; UAS-RNAi-Parp/elavGS

10.1.2 Protein Extraction

For extraction of flies protein, the heads of the corresponding genotypes of flies were isolated and added to RIPA lysis buffer (50 mM Tris pH 8.0, 150 mM NaCl, 1% NP-40, 5 mM EDTA, 0.5% sodium deoxycholate, 0.1% SDS, protease inhibitor) and fully ground on ice. The lysate was centrifuged at high speed at four degrees and the supernatant was added to 4×LDS sample buffer (Invitrogen) and boiled at 95° C. For obtaining insoluble phase, the precipitate obtained by centrifugation after lysis of RIPA was lysed with 9 M urea buffer (9 M urea, 50 mM Tris buffer, pH 8.0) equal to the soluble volume, then 4×LDS sample buffer (Invitrogen) was added and boiled at 95° C. for subsequent research.

10.1.3 qPCR Detection

For extraction of Drosophila mRNA, the heads of the corresponding genotypes of Drosophila were isolated and added to Trizol (Invitrogen), then fully ground with a grinding pestle, followed by chloroform extraction and isopropanol precipitation to obtain mRNA, and then DNase (Promega) was added to eliminate the contamination of genomic DNA. 1 μg of mRNA was taken for reverse transcription using High-Capacity cDNA Reverse Transcription Kit (Applied biosystems). The obtained cDNA was mixed with SYBR Green qPCR Master Mix (Bimake) and target gene primers by a certain ratio and then subjected to qPCR assay (QuantStudio™ 6 Flex Real-Time PCR system (Life Technologies)). All primers used are as follows:

dParp:
Upstream primer:
(SEQ ID NO: 23)
5′-ATGAAGTACGGAGGCCAACC-3′;
Downstream primer:
(SEQ ID NO: 24)
5′-TCTTCACCTGACGCAAACCA-3′
dActin:
Upstream primer:
(SEQ ID NO: 25)
5′-GAGCGCGGTTACTCTTTCAC-3′;
Downstream primer:
(SEQ ID NO: 26)
5′-GCCATCTCCTGCTCAAAGTC-3′

10.1.4 Test of external eye injury, crawling ability and survivability of Drosophila

Observation of the external eyes of Drosophila: Drosophila of the corresponding genotypes were observed under an SZX16 (Olympus) microscope at 5 and 20 days after fledge and pictures of their external eyes were taken. Each Drosophila was classified as 5 classes of “0, 1, 2, 3, 4” according to the score of damage on its external eye, where “0” represents no damage at all and “4” represents complete damage. Each Drosophila participated in the follow-up statistics as an independent sample.

Test of Drosophila crawling ability: On the first day of fledging, Drosophila of the corresponding genotype were selected, and Drosophila were fed in food containing 80 μg/ml RU486 (TCI) to induce transgene expression in groups of 20. On the specified date, Drosophila were divided into 5-8 transparent plastic tubes in groups of 20, and the tubes were gently shaken to make the Drosophila stay at the bottom of the tubes and timing was started. The number of Drosophila that could climb over 3 cm height within 10 s was recorded and the percentage was calculated (the whole Drosophila in each tube was taken as an independent sample for subsequent counting, the same below). All experiments were performed in triplicate.

Test of Drosophila survivability: On the first day of fledging, Drosophila of the corresponding genotype were selected and divided into 7-9 tubes in groups of 20, and fed in food containing 80 μg/ml RU486 (TCI). All Drosophila used for viability test were fed with fresh food that was replaced every three days for adding the drug. The number of the Drosophila that die in each tube was recorded each time, and the survival rate of the Drosophila was calculated (the Drosophila that flew away accidentally were not counted in the total).

10.2. Experimental Results (as Shown in FIG. 10)

(A-E) Knockdown of PARP in Drosophila can alleviate the optic nerve degeneration caused by the transgenic TDP-43. (A) The picture shows the external eye of a single representative Drosophila in the experiment. Expression in the photoreceptor nerves of Drosophila with the GMR driver (mating Drosophila strains according to the genotypes listed, the transgenic sequences can be automatically expressed in specific cells of the offspring Drosophila. This step can be done in accordance with the conventional molecular genetics in the art. Human TDP-43 protein (mated with Drosophila strains according to the listed genotypes, and the transgene encoding human TDP-43 protein can be used for expression in Drosophila; TDP-43 protein sequence is shown in SEQ ID NO: 2) will cause obvious optic nerve degeneration, mainly manifested by the loss of pigment and the mattness of the outer eye surface, and this phenotype will exacerbated with the aging of the Drosophila (Day5 vs Day20). Simultaneous expression of RNAi-Parp in the transgenic Drosophila (the same as above, mating fruit flies according to the listed genotypes, the transgenic sequences can be automatically expressed in specific cells of the offspring Drosophila) can well alleviate the neurodegenerative disease caused by TDP-43. The figure in the upper left corner is an enlarged area in the white box. (B) Statistics of the score of damage to the external eye in (A). The numbers in parentheses represent the number of Drosophila involved in the statistics. (C) qPCR assay of mRNA levels of Parp in the head of RNAi-Parp transgenic Drosophila. (D) The figure shows that knockdown of Parp has no significant effect on exogenous protein expression and solubility in TDP-43 transgenic Drosophila which was verified with WB. Wherein GAPDH was used as an internal reference to demonstrate the consistent protein loading of the two groups of samples and there was no contamination of the soluble phase proteins in the insoluble phase. (E) Statistics of the relative expression of TDP-43 protein in figure (D), where “ud” means that no protein was detected.

(F-G) Knockdown of PARP in adult Drosophila neurons ameliorates the crawling deficit and shortened lifespan induced by expression of human-derived TDP-43 (G) Induction of expression of human-derived TDP-43 transgene in adult Drosophila neurons driven by elavGS caused age-dependent loss of crawling ability, while concomitant knockdown of PARP alleviates this phenotype. (F) The lifespan of Drosophila under the same conditions was significantly shorter compared with the control group, and knocking down PARP can extend the lifespan of transgenic Drosophila to some extent. The numbers in parentheses represent the number of Drosophila and the half survival days of the final count, respectively.

All experiments in FIG. 10 were performed in triplicate. The significance of statistical differences was determined by the unpaired, two-tailed Student's t-test method to test the p value, where ns represents no significant difference between the two groups;** is p<0.01 and *** is p<0.001.

The results of Example 10 show that the reduction of PARPase levels has a significant ameliorative effect on inhibiting the degenerative neuronal morphology, function and even the dramatic shortening of life span caused by TDP-43 overload, not only in in vitro and cellular assays, but also in animal models of ALS in vivo and at the overall level of the living organism, suggesting that the development of PARP inhibitors is promising to treat ALS.

The PARP inhibitor Olaparib in the present invention is a clinical drug approved by the FDA for the treatment of breast cancer, ovarian cancer, prostate cancer and other tumors (Fong et al., 2009; Tuft et al., 2010), and its side effects have been clinically tested. At the same time, the present invention shows that the inhibitor has a significant inhibitory effect on both hnRNP Al and TDP-43-induced neurocytotoxicity, so it is more likely and less risky to be used for the development of clinical drugs for ALS.

Although the above describes specific embodiments of the present invention, it should be understood by those skilled in the art that these are merely illustrative examples and that a variety of changes or modifications can be made to these embodiments without departing from the principles and substance of the present invention. Therefore, the scope of protection of the invention is limited by the appended claims.

1. A pharmaceutical composition for treatment of neurodegenerative diseases, wherein the pharmaceutical composition comprises a drug that reduces level of intracellular poly(ADP-ribosyl)ation, preferably a drug that reduces intracellular level of poly(ADP-ribosyl)ation of RNA-binding proteins, wherein the RNA-binding proteins are preferably hnRNP A1 or TDP-43.

2. The pharmaceutical composition for treatment of neurodegenerative diseases of claim 1, wherein the neurodegenerative disease is amyotrophic lateral sclerosis.

3. A pharmaceutical composition for treatment of diseases caused by abnormality of RNA-binding proteins, wherein the pharmaceutical composition comprising a drug that reduces intracellular level of poly(ADP-ribosyl)ation.

4. The pharmaceutical composition for treatment of diseases caused by abnormality of RNA-binding proteins of claim 3, wherein the treatment of diseases caused by abnormality of RNA-binding proteins comprises one or more of:
(a) diseases caused by abnormal intracellular aggregation resulting from abnormal post-translational modification of RNA-binding protein;
(b) diseases caused by abnormal subcellular localization of RNA-binding protein;
(c) diseases caused by abnormality of formation or regulation of stress granules in which RNA-binding proteins are involved.

5. The pharmaceutical composition for treatment of diseases caused by abnormality of RNA-binding proteins of claim 3, wherein the treatment of diseases caused by abnormality of RNA-binding proteins comprises one or more of:
(a) diseases caused by abnormality of RNA-binding protein binding or regulating processing, shearing, transportation or translation of RNA;
(b) diseases caused by interaction between the RNA-binding protein such as hnRNP A1 and other protein like TDP-43 protein or subcellular localization of the RNA-binding protein such as hnRNP A1, which is affected by covalent poly(ADP-ribosyl)ation of RNA-binding proteins such as hnRNP A1;
(c) diseases caused by interaction between the RNA-binding proteins such as hnRNP A1 and other proteins such as TDP-43 protein, which is affected by non-covalent binding of the RNA-binding proteins such as hnRNP A1 to PAR;
(d) diseases caused by transport of PAR to stress granules under cellular stress conditions, which is affected by non-covalent binding of the RNA-binding proteins to PAR; or
(e) diseases caused by destruction of cell homeostasis due to change of solubility of cellular PAR, which is affected by non-covalent binding of the RNA-binding proteins to PAR.

6. The pharmaceutical composition for treatment of diseases caused by abnormality of RNA-binding proteins of claim 3, wherein the drug capable of reducing intracellular level of poly(ADP-ribosyl)ation is a drug capable of increasing expression level of PARG hydrolase or reducing expression level of PARP polymerase.

7. The pharmaceutical composition for treatment of diseases caused by abnormality of RNA-binding proteins of claim 6, wherein the drug capable of increasing level of expression of PARG hydrolase is a PARG hydrolase agonist; the drug capable of reducing expression level of PARP polymerase is a PARP polymerase inhibitor.

8. A method of treating a neurodegenerative disease, wherein the method comprises treatment using the pharmaceutical composition of claim 1.

9. The method of claim 8, wherein the pharmaceutical composition provides treatment through one or more of the following mechanisms:
(a) Affecting hnRNP A1 or TDP-43 to form stress granules, preferably inhibiting assembly of stress granules or promoting disassembly of stress granules;
(b) Affecting interaction between hnRNP A1 and other poly(ADP-ribosyl)ation proteins;
(c) Affecting interaction between hnRNP A1 and other PAR binding proteins;
(d) Affecting subcellular localization or stress response of hnRNP A1;
(e) Affecting liquid-liquid phase separation or aggregation tendency of hnRNP A1;
(f) Affecting co-phase separation of hnRNP A1 and TDP-43 protein;
(g) Affecting intracellular interaction between hnRNP A1 and TDP-43 protein; or
(h) Inhibiting neurocytotoxicity caused by hnRNP A1 or TDP-43.

10. The method of claim 9, wherein the pharmaceutical composition can ultimately inhibit neurodegeneration caused by hnRNP A1 or TDP-43.

11.-12. (canceled)

13. The pharmaceutical composition for treatment of diseases caused by abnormality of RNA-binding proteins of claim 3, wherein the pharmaceutical composition comprising a drug that reduce level of intracellular poly(ADP-ribosyl)ation of the RNA-binding proteins.

14. The pharmaceutical composition for treatment of diseases caused by abnormality of RNA-binding proteins of claim 13, wherein the RNA-binding proteins are hnRNP A1 or TDP-43.

15. The pharmaceutical composition for treatment of diseases caused by abnormality of RNA-binding proteins of claim 7, wherein the PARP polymerase inhibitor is Olaparib.

16. The method of claim 10, wherein the neurodegeneration comprises: 1) inhibition of toxicity in motor neuron like NSC-34 cells, or 2) alleviation of neuron degeneration, declined motor ability and/or shortened life span in Drosophila model of ALS.

17. A method of treating a neurodegenerative disease, wherein the method comprises treatment using the pharmaceutical composition of claim 3.

18. A method of treating a disease caused by abnormality of RNA-binding proteins, wherein the method comprises treatment using the pharmaceutical composition of claim 1.

19. A method of treating a disease caused by abnormality of RNA-binding proteins, wherein the method comprises treatment using the pharmaceutical composition of claim 3.
